(19) United States
(12) Patent Application Publication
(10) Pub. No.: US 2008/0089859 A1
(43) Pub. Date: Apr. 17, 2008

(54) ADMINISTRATION OF SLEEP RESTORATIVE AGENTS

(76) Inventor: Andrew Holman, Seattle, WA (US)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(21) Appl. No.: 11/736,406

(22) Filed: Apr. 17, 2007

Related U.S. Application Data
(63) Continuation of application No. 10/091,744, filed on Mar. 5, 2002, now abandoned.
(60) Provisional application No. 60/273,667, filed on Mar. 5, 2001.

(51) Int. Cl. A61K 31/428; A61K 31/196; A61K 31/404; A61K 31/437; A61K 31/5025; A61K 31/52; A61K 31/56; A61K 38/00; A61K 38/21; A61P 37/06; A61P 25/20; A61K 39/395; A61K 38/13; A61K 31/675; A61K 31/5513; A61K 31/519; A61K 31/4427; A61K 31/433; A61K 31/42

(52) U.S. Cl. 424/85.4; 424/143.1; 514/110; 514/12; 514/171; 514/221; 514/249; 514/259.3; 514/263.2; 514/291; 514/300; 514/361; 514/367; 514/378; 514/418; 514/561; 514/9

(57) ABSTRACT

The present invention provides methods and compositions for increasing the efficacy of a therapeutic agent administered to a subject. A steep restorative agent is co-administered to the subject along with the therapeutic agent, whereby the efficacy of the therapeutic agent is increased.

This application claims the benefit of U.S. Provisional Patent Application No. 60/273,667, filed Mar. 5, 2001.

### BACKGROUND OF THE INVENTION

Therapeutic agents are used to treat a wide variety of conditions in patients. The effectiveness of therapeutic agents can vary in patients, however, depending on a number of factors, such as, for example, the genetic makeup of patient, the bioavailability of the drug in the patient, and the ability of the drug to reach the target cells or tissue. Despite positive indications for these factors, some patients do not respond to otherwise effective therapeutic agents.

For example, many drug options are available to treat inflammatory arthritis, and most drugs decrease synovitis and joint destruction by inhibiting lymphocyte function. Tumor necrosis factor alpha (TNFα) is one soluble factor responsible for inflammatory arthritis. TNFα binds to its receptor (TNFα receptor) and participates in the inflammatory response associated with the immunological recognition of infectious agents. The inflammatory response plays an important role in limiting and controlling pathogenic infections.

Elevated levels of TNFα are believed to cause or exacerbate inflammatory arthritis. For example, rheumatoid synovial tissue becomes invaded with inflammatory cells that result in destruction to cartilage and bone. Early in the destructive process, macrophages identify an offending antigen, then initiate the inflammatory cascade by secreting TNFα to draw additional T lymphocytes into the joint and surrounding tissue. Blocking TNFα signaling of this process decreases the inflammatory cascade and deters destruction. If TNFα is blocked, the secondary cells will not secrete other cytokines, including interleukin-1, which plays a pivotal role in bone erosion formation.

A soluble form of the TNFα receptor has been engineered as a therapeutic agent to treat inflammatory arthritis. The soluble receptor binds to soluble TNFα and reduces its concentration in vivo. One version of the soluble TNFα receptor, Etanercept, is sold under the trademark ENBREL® (Immunex, Seattle, Wash.). Etanercept is a dimeric fusion protein of the extracellular ligand-binding domain of the p75 TNFα receptor linked to an Fc portion of human IgG1. (See, e.g., Breedveld, Eur. Cytokine Netw. 9:233-38 (1998); Mohler et al., J. Immunol. 151:1548-61 (1993).) Another version of the TNFα receptor, Lenercept (also called Ro 45-2081; Hoffian-LaRoche Inc., Nutley, N.J.), has demonstrated efficacy in various animal models of allergic lung inflammation and acute lung injury. Lenercept is a recombinant chimeric molecule constructed from the soluble 55 kDa human TNF receptor fused to the hinge region of the heavy chain IgG1 gene (Renzetti et al., Inflamm. Res. 46:S143 (1997)).

Etanercept has proved markedly successful for a wide variety of patient who have severe arthritis. Subsequent research revealed Etanercept efficacy for many forms of inflammatory arthritis, including psoriatic arthritis and ankylosing spondylitis. Unfortunately, 15-20% of patients with rheumatoid arthritis may not respond to Etanercept. Thus, despite the overall remarkable efficacy for many patients, an explanation for this inconsistent response is not understood. Current theories suggest that TNFα may not be the primary cytokine for all rheumatoid arthritis patients, or that rheumatoid arthritis may be a more inhomogeneous disease than initially thought.

Other therapeutic agents show a similar non-efficacious response in some patients. For example, other diseases modifying agents (DMARDs), such as corticosteroids, hydroxychloroquine, sulfasalazine, methotrexate, gold, penicillamine, azathioprine, cyclosporine, cyclophosphamide, leflunamide, and Infliximab, are effective in many patients, but do not decrease synovitis, or control inflammatory disease activity and joint destruction, effectively in all patients. Thus, there remains a need for compositions and methods for increasing the efficacy of therapeutic agents in patients. The present invention satisfies this need and other needs.

### BRIEF SUMMARY OF THE INVENTION

The present invention provides methods for increasing the efficacy of a therapeutic agent administered to a subject in need thereof by administering to the subject an effective amount of a sleep restorative agent or a pharmacologically acceptable addition salt thereof. The sleep restorative agent can also reduce undesired side effects associated with administration of the therapeutic agent, reduce symptoms of the subject, and/or increase sleep quality in the subject. In one embodiment, administration of the sleep restorative agent spares the effective amount of the therapeutic agent. Increased sleep quality by the subject can be manifested as, for example, restoration or prolongation of stage III/IV sleep, decreased sleep fragmentation or disruption, sleep apnea, restless legs syndrome, restlessness, racing thoughts, talking in one's sleep, teeth grinding, nightmares, and the like. In additional embodiments, the sleep restorative agent can reduce increased or excessive sympathetic tone in a subject.

The sleep restorative agent can be a tetrahydro-benzthiazole compound, such as, for example, 2-amino-4,5,6,7-tetrahydro-6-(propylamino)benzo-thiazole or the (−)-enantiomer thereof. The sleep restorative agent can also be a 3(H) indolone, such as, for example, 4-[2-(dipropylamino)-ethyl]-1,3-dihydro-2H-indol-2-one, or Lorazepam, Clonazepam, Tizanidine, Gabapentin, Zaleplon, Zolpidem, or pharmaceutically acceptable salts thereof.

The therapeutic agent can be, for example, an immunomodulatory agent, such as a soluble TNFα receptor, methotrexate, prednisone, an interferon, a cyclosporin, an ascomycin, a rapamycin, a corticosteroid, a cyclophosphamide, azathioprine, brequinar, leflunamide, mizoribine, deoxyspergualin, immunosuppressive monoclonal antibodies to a leukocyte receptor, and the like. The sleep restorative agent and the therapeutic agent can be administered in a unitary dosage form, or administered separately. Suitable dosage forms include, for example, tablets, capsules, lozenges, powders, solutions, suspensions, emulsions, injectable solutions, syrups, suppositories, transdermal patches, and the like. The compositions can also be admixed with a pharmaceutically acceptable carrier.

In another aspect, methods are provided for sparing an effective amount of a therapeutic agent administered to a subject having an autoimmune condition by co-administering to the subject the therapeutic agent and an effective amount of a sleep restorative agent, the sleep restorative agent improving sleep quality of the patient so that the sleep restorative agent spares the effective amount of the therapeutic agent. In one embodiment, administration of the sleep restorative agent can reduce an undesired side effect associated with administration of the therapeutic agent. The autoimmune condition can be, for example, rheumatoid arthritis; psoriatic arthritis; a spondyloarthropathy; palindromic rheumatism; systemic lupus erythematosus; vasculitis with systemic lupus erythematosus; multiple sclerosis; Hashimoto's thyroiditis; chronic pseudogout; hepatitis C arthritis, mixed connective tissue disease; dermatomyositis, polymyositis; scleroderma; Sjogren's syndrome; cryoglobulinemia; Crohn's disease; ulcerative colitis; autoimmune hepatitis; sclerosing cholangitis; primary biliary cirrhosis; autoimmune pneumonitis; autoimmune cerebritis; thyroiditis; graft versus host disease; Myasthenia gravis; pemphigus vulgaris; temporal arteritis; polymyalgia rheumatica; autoimmune hemolytic anemia; idiopathic thrombocytopenic purpura; thrombotic thrombocytopenic purpura; hemolytic uremic syndrome; Sweet's syndrome; polyarteritis nodosa; microscopic polyarteritis nodosa; amyloidosis; sarcoidosis; familial Mediterranean fever; and the like. The spondyloarthropathy can be, for example, Behcet's disease, sarcoidosis, ankylosing spondylitis, Whipple's Disease or Reiter's Syndrome.

In another aspect, methods are provided for reducing a symptom in a subject in need of immunomodulatory therapy by co-administering an effective amount of an immunomodulatory agent and an effective amount of a sleep restorative agent, the sleep restorative agent improving sleep quality of the subject. The sleep restorative agent typically spares the effective amount of the immunomodulatory agent needed to reduce the symptom. The immunomodulatory agent can be, for example, soluble TNFα receptor, methotrexate, an interferon, a cyclosporin, an ascomycin, a rapamycin, prednisone, other corticosteroids, a cyclophosphamide, azathioprine, brequinar, leflunamide, mizoribine, deoxyspergualin, immunosuppressive monoclonal antibodies to a leukocyte receptor, and the like. In some embodiments, the subject has a sleep disorder. Administration of the sleep restorative agent can reduce an undesired side effect associated with administration of the therapeutic agent.

In another aspect, compositions for administration to a subject having an autoimmune disease are provided. The compositions typically include an effective amount of a sleep restorative agent, and an effective amount of a therapeutic agent. The effective amount of the sleep restorative agent typically spares the effective amount of the therapeutic agent. The composition can optionally be administered as a unitary dose, and can be a tablet, capsule, lozenge, powder, solution, suspension, emulsion, injectable solution, syrup, suppository, transdermal patch, and the like. The composition can optionally further include a pharmaceutically acceptable carrier, an excipient, an adjuvant, and the like.

The sleep restorative agent can be, for example, a tetrahydro-benzthiazole compound, such as, for example, 2-amino-4,5,6,7-tetrahydro-6-(propylamino)benzo-thiazole or the (−)-enantiomer thereof, a 3(H) indolone, such as, for example, 4-[2-(dipropylamino)-ethyl]-1,3-dihydro-2H-indol-2-one, or Lorazepam, Clonazepam, Tizanidine, Gabapentin, Zaleplon, Zolpidem, pharmaceutically acceptable salts thereof, and the like. The therapeutic agent can be, for example, soluble TNFα receptor, methotrexate, prednisone, other corticosteroids, an interferon, a cyclosporin, an ascomycin, a rapamycin, a cyclophosphamide, azathioprine, brequinar, leflunamide, mizoribine, deoxyspergualin, an immunosuppressive monoclonal antibodies to a leukocyte receptor, and the like.

### DESCRIPTION OF THE SPECIFIC EMBODIMENTS

The present invention provides methods and compositions for increasing the efficacy of a therapeutic agent administered to a subject (e.g., a human patient). An agent according to the present invention can be coadministered to the subject with the therapeutic agent, whereby the efficacy of the therapeutic agent is increased. The efficacy of the therapeutic agent can be increased, for example, by decreasing the amount of the therapeutic agent required to be effective in the subject (i.e., decreasing the effective amount), by reducing, or further reducing, one or more symptoms in the subject, by improving the sleep quality of the subject by reducing excessive sympathetic tone, and the like. In certain embodiments, administration of the agent spares the amount of the therapeutic agent that is administered to achieve a comparable reduction of symptom(s), as compared with a subject receiving the therapeutic agent alone (i.e., without the sleep restorative agent).

Agents

In one aspect, the invention provides agents and methods of using such agents to increase the efficacy of a therapeutic agent. The agent can be an agent that improves the sleep quality of the subject (e.g., a sleep restorative agent). For example, a sleep restorative agent can restore or prolong stage III/IV sleep in the subject, reduce sleep fragmentation or disruption (i.e., frequent waking during sleep), sleep apnea, restless, racing thoughts, talking in one's sleep, nightmares, teeth grinding, restless legs syndrome, and the like.

In related embodiments, sleep restorative agents can decrease sympathetic tone, or decrease increased sympathetic tone, in the subject. The sympathetic nervous system responds to environmental and endogenous stresses to maintain homeostasis for a wide variety of basic physiologic functions, such as, for example, thermogenesis, regional blood flow, bowel motility, gastric acidity, blood pressure, heart rate, sweat glands, and sleep. Increased sympathetic tone can lead to increased, perspiration, gastric acidity, bowel motility, heart rate, temperature and blood flow through vascular tone blood pressure, or disruption of deep sleep. Excessive or increased sympathetic tone is typically chronically observed in the subject (e.g., over a period of weeks or months or longer). Administration of a sleep restorative agent according to the present invention can reduce excessive or increased sympathetic tone in the subject (e.g., chronic or persistent elevated sympathetic tone), and/or increase the frequency and/or duration of deep sleep.

In certain embodiments, the sleep restorative agent can be a non-ergot, dopamine agonist, such as, for example, a D2/D3 dopamine agonist, such as a tetrahydro-benzthiazole compound of the following formula I:

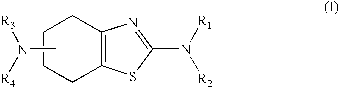

wherein

R₁ represents a hydrogen atom, a C₁₋₆ alkyl group, a C₃₋₆ alkenyl, a C₃₋₆ alkynyl group, a C₁₋₆ alkanoyl group, a phenyl C₁₋₃ alkyl group, or a phenyl C₁₋₃ alkanoyl group, wherein the phenyl nuclei can be substituted by halogen atoms (e.g. 1 or 2);

R₂ represents a hydrogen atom or a C₁₋₄ alkyl group;

R₃ represents a hydrogen atom, a C₁₋₇ alkyl group, a C₃₋₇ cycloalkyl group, a C₁₋₃ alkenyl, a C₁₋₃ alkynyl group, a C₁₋₇ alkanoyl group, a phenyl C₁₋₃ alkyl, or a phenyl C₁₋₃ alkanoyl group, wherein the phenyl nuclei can be substituted by fluorine, chlorine and/or bromine atoms;

R₄ represents a hydrogen atom, a C₁₋₄ alkyl group, a C₃₋₆ alkenyl, or a C₃₋₆ alkynyl group, or R₃ and R₄ together with the nitrogen atom between them represent a pyrrolidino, piperidino, hexamethyleneimino or morpholino group;

and the pharmacologically acceptable acid addition salts thereof, alone or in association with a pharmaceutically acceptable carrier.

The tetrahydro-benzthiazole compounds of general formula (I) can be those wherein the R₃-R₄ group is in the 5- or 6-position. Examples of the R₃-R₄ amino group include amino, methylamino, ethylamino, n-propylamino, isopropylamino, n-butylamino, isobutylamino, tert-butylamino, n-pentylamino, isoamylamino, n-hexylamino, n-heptylamino, dimethylamino, diethylamino, di-n-propylamino, di-n-butylamino, methyl-ethylamino, methyl-n-propylamino, methyl-isopropylamino, ethyl-isopropylamino, allylamino, buten-2-ylamino, hexen-2-ylamino, diallylamino, N-methyl-allylamino, N-ethyl-allylamino, N-n-propyl-allylamino, N-n-butyl-allylamino, propargylamino, butin-2-ylamino, hexin-2-ylamino, dipropargylamino, N-methyl-propargylamino, N-ethyl-propargylamino, cyclopropylamino, cyclobutylamino, cyclopentylamino, cyclohexylamino, cycloheptylamino, N-methyl cyclohexylamino, N-ethyl-cyclohexylamino, formylamino, acetylamino, propionylamino, butanoylamino, pentanoylamino, hexanoylamino, heptanoylamino, N-methyl-acetylamino, N-ethyl-acetylamino, N-n-propyl-acetylamino, N-allyl-acetylamino, benzoylamino, fluorobenzoylamino, chlorobenzoylamino, bromobenzoylamino, phenylacetamino, 2-phenylpropionylamino, N-methyl-benzoylamino, N-ethyl-chlorobenzoylamino, dichlorobenzoylamino, N-cyclohexyl-acetylamino, benzylamino, chlorobenzylamino, bromobenzylamino, 1-phenylethylamino, 2-phenylethylamino, 2-phenyl-n-propylamino, 3-phenyl-n-propylamino, N-methyl-benzylamino, N-ethyl-benzylamino, N-ethyl-chlorobenzylamino, N-ethyl-2-phenylethylamino, N-acetyl-benzylamino, N-acetyl-chlorobenzylamino, N-allyl-benzylamino, N-allyl-chlorobenzylamino, pyrrolidino, piperidino, hexamethyleneimino or morpholino group.

The R₁-R₂ amino group can be, for example, amino, methylamino, ethylamino, n-propylamino, isopropylamino, n-butyl amino, isobutylamino, tert-butylamino, n-pentylamino, isoamylamino, n-hexylamino, dimethylamino, diethylamino, di-n-propylamino, di-n-butylamino, methyl-ethylamino, methyl-n-propylamino, methyl-isopropylamino, ethyl-isopropylamino, allylamino, buten-2-ylamino, hexen-2-ylamino, N-methyl-allylamino, N-ethyl-allylamino, N-n-propyl-allylamino, N-n-butyl-allylamino, propargylamino, N-methyl-propargylamino, N-n-propyl-propargyl amino, formylamino, acetylamino, propionylamino, butanoylamino, hexanoylamino, N-methyl-acetylamino, N-allyl-acetylamino, N-propargyl-acetylamino, benzylamino, N-methyl-benzylamino, 2-chloro-benzylamino, 4-chloro-benzylamino, 4-fluoro-benzylamino, 3,4-dichloro-benzylamino, 1-phenylethylamino, 2-phenylethylamino, 3-phenyl-n-propylamino, benzoylamino phenacetylamino or 2-phenylpropionylamino group.

In additional embodiments, the sleep restorative agents of general formula (I) can be compounds of the following general formula (Ia):

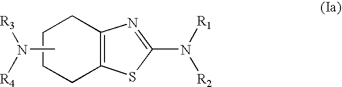

wherein

R₁ represents a hydrogen atom, an alkyl group having 1 to 3 carbon atoms, an allyl, benzyl, 2-chloro-benzyl, 4-chloro-benzyl, 3,4-dichloro-benzyl or phenylethyl group;

R₂ represents a hydrogen atom, a methyl or ethyl group;

R₃ represents a hydrogen atom, an alkyl group with 1 to 6 carbon atoms, an allyl, propargyl, benzyl, chlorobenzyl, phenylethyl, cyclopentyl or cyclohexyl group;

R₄ represents a hydrogen atom, an alkyl group having 1 to 3 carbon atoms or an allyl group; or

R₃ and R₄ together with the nitrogen atom between them represent a pyrrolidino, piperidino, hexamethyleneimino or morpholino group,

The R₃-R₄ amino group can be in the 6-position, The sleep restorative agents of formula (Ia) can also be an acid addition salt, such as pharmaceutically acceptable addition salts, either alone or together with a pharmaceutically acceptable carrier.

In additional embodiments, the tetrahydro-benzthiazole is a compound of the following formula (Ib):

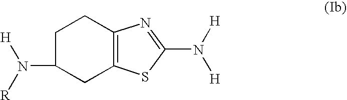

wherein

R is a hydrogen atom, a C₁₋₇ alkyl group, a C₃₋₇ cycloalkyl group, a C₃₋₆ alkenyl, a C₃₋₆ alkynyl group, or a phenyl C₁₋₃ alkyl group, wherein the phenyl nucleus can be substituted by fluorine, chlorine or bromine atoms, or a pharmaceutically acceptable acid addition salt thereof.

In an exemplary embodiment, the sleep restorative agent is pramipexole, such as a pharmaceutical formulation of (S)-2-amino-4,5,6,7-tetrahydro-6-(propylamino)benzo-thiazole dihydrochloride monohydrate available from Pharmacia & Upjohn under the trademark MIRAPEX®.

The synthesis, formulation and administration of the tetrahydro-benzthiazole compounds of formula (I) according to the present invention are described in, for example, U.S. Pat. Nos. 4,843,086; 4,886,812; 5,112,842; 5,650,420 and 6,001,861 (the disclosures of which are incorporated by reference herein). The compounds of general formula (I) have at least one chiral center and can, therefore, exist in the form of various stereoisomers. The invention embraces all of these stereoisomers and mixtures thereof. Mixtures of these stereoisomers can be resolved by conventional methods, such as, for example, by column chromatography on a chiral phase, by fractional crystallization of the diastereomeric salts or by column chromatography of their conjugates with optically active auxiliary acids such as tartaric acid, O,O-dibenzoyl-tartaric acid, camphor acid, camphorsulfonic acid or α-methoxy-phenylacetic acid.

In additional embodiments, the sleep restorative agent can also be a 3(H)-indolone of the following formula II:

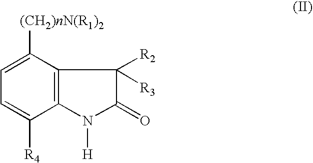

wherein each of R₁, R₂ and R₃ are each independently hydrogen or C₁₋₄ alkyl; R₄ is hydrogen or hydroxy; and n is 1 to 3; or a pharmaceutically acceptable salt thereof, alone or in association with a pharmaceutically acceptable carrier.

In another embodiment, the 3(H)-indolone compounds of general formula (II) can be those wherein the group R₁ is C₁₋₄ alkyl, such as propyl, R₂ and R₃ are both hydrogen, and R₄ is hydrogen or hydroxy. For example, the 3(H)-indolone compounds can be a compound of formula (II) in which R₁ is propyl, R₂, R₃ and R₄ are hydrogen and n is 2, namely the compound 4-[2-(dipropylamino)-ethyl]-1,3-dihydro-2H-indol-2-one, or a pharmaceutically acceptable salt thereof. Suitable salts include, for example, acid addition salts, such as hydrochloride addition salts.

In an exemplary embodiment, the sleep restorative agent can be a selective dopamine D2 receptor agonist, such as Ropinirole, which is a pharmaceutical formulation of 4-[2-(dipropylamino)-ethyl]-1,3-dihyro-2H-indol-2-one available from Smith Kline Beecham under the trademark Requip®.

The synthesis, formulation and administration of the 3(H)-indolone compounds of formula (II) above are described in, for example, U.S. Pat. No. 4,452,808, the disclosure of which is incorporated by reference herein.

The steep restorative agent, such as the compounds of formula (I) or (II), can be formulated as a pharmaceutically acceptable salt and can optionally further include a pharmaceutically acceptable carrier. The compounds of formula (I) and (II) can also be converted into the acid addition salts thereof, particularly the pharmaceutically acceptable acid addition salts with inorganic or organic acids. Suitable acids for this include, for example, hydrochloric, hydrobromic, sulfuric, phosphoric, lactic, citric, tartaric, succinic, maleic or fumaric acid.

The sleep restorative agent also can be night-time anti-depressant class medication and/or a muscle relaxant, such as, for example, Lorazepam, Clonazepam, alone or in combination with Trazodone, Carisoprodol, other muscle relaxants, and/or melatonin. In other embodiments, the sleep restorative agent can be Lorazepam or Clonazepam with Pramipexole, Ropinirole (e.g., Requip®) or Pergolide (e.g., PERMAX®), with or without Trazodone, other night time anti-depressant class medications, a muscle relaxant and/or melatonin.

The sleep restorative agent can also be Tizanidine (which is sold under the trademark ZANAFLEX®), alone or administered with Pramipexole, Ropinole, Lorazepam or Clonazepam. Trazodone, other night time anti-depressant class medications, Carisoprodol, or other muscle relaxants or melatonin can optionally be co-administered.

The sleep restorative agent can also be Gabapentin (which is sold under the trademark NEURONTIN®), pregabalin or Milnacipran (a norepinephrine serotonin reuptake inhibitor). SINEMET® (Sinemet CR, which is a sustained-release tablet containing a mixture of Carbidopa and Levodopa, available from The DuPont Merck Pharmaceutical Co.), Zolpidem (e.g., AMBIEN®), Zaleplon (e.g., SONATA®), valarian root, selective serotonin reuptake inhibitors (SSRI's), serotonin uptake inhibitor, and elemental magnesium can also be used as sleep restorative agents.

The sleep restorative agents according to the present invention can be used in the form of salts derived from inorganic or organic acids. These salts can include, but are not limited to, the following: acetate, adipate, alginate, citrate, aspartate, benzoate, benzenesulfonate, bisulfate, butyrate, camphorate, camphorsulfonate, digluconate, cyclopentanepropionate, dodecylsulfate, ethanesulfonate, glucoheptanoate, glycerophosphate, hemisulfate, heptanoate, hexanoate, fumarate, hydrochloride, hydrobromide, hydroiodide, 2-hydroxyethanesulfonate, lactate, maleate, methanesulfonate, nicotinate, 2-napthalene-sulfonate, oxalate, pamoate, pectinate, persulfate, 3-phenylpropionate, picrate, pivalate, propionate, succinate, tartrate, thiocyanate, p-toluenesulfonate and undecanoate. Also, basic nitrogen-containing groups can be quaternized with such agents as lower alkyl halides, such as methyl, ethyl, propyl, and butyl chloride, bromides, and iodides; dialkyl sulfates like dimethyl, diethyl, dibutyl, and diamyl sulfates; long chain halides such as decyl, lauryl, myristyl and stearyl chlorides, bromides and iodides; aralkyl halides like benzyl and phenethyl bromides; and the like. Water or oil-soluble or dispersible products are thereby obtained.

Examples of acids which can be employed to form pharmaceutically acceptable acid addition salts include such inorganic acids as hydrochloric acid, sulphuric acid and phosphoric acid and such organic acids as oxalic acid, maleic acid, succinic acid and citric acid. Basic addition salts can be prepared in situ during the final isolation and purification, or separately by reacting carboxylic acid moieties with a suitable base such as the hydroxide, carbonate or bicarbonate of a pharmaceutical acceptable metal cation or with ammonia, or an organic primary, secondary or tertiary amine. Pharmaceutical acceptable salts include, but are not limited to, cations based on the alkali and alkaline earth metals, such as sodium, lithium, potassium, calcium, magnesium, aluminum salts and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. Other representative organic amines useful for the formation of base addition salts include diethylamine, ethylenediamine, ethanolamine, diethanolamine, piperazine and the like.

Therapeutic Agents

The therapeutic agent can be any agent used to treat a disease or condition in a subject. As used herein, the term “therapeutic agent” broadly refers to a drug or other compound that is administered to a subject to reduce or alleviate on or more symptoms of a disease or condition. The sleep restorative agent and the therapeutic agent are typically different (e.g., the therapeutic agent is not a sleep restorative agent).

In certain embodiments, the disease or condition can be an “immune condition,” which generally refers to a disease or condition which is exacerbated by an immune response of a subject. The immune condition can be, for example, an “autoimmune condition,” which refers to a disease or condition in which a subject's own immune cells or antibodies are directed against cells or tissues of the subject. Autoimmune conditions include, for example, Type I diabetes; multiple sclerosis; systemic lupus erythematosis (SLE); rheumatoid arthritis; psoriatic arthritis; a spondyloarthropathy, including Behcet's disease, sarcoidosis, ankylosing spondylitis, Whipple's Disease, and Reiter's Syndrome; palindromic rheumatism; vasculitis with SLE, Hashimoto's thyroiditis; chronic pseudogout; hepatitis C arthritis; mixed connective tissue disease; dermatomyositis; polymyositis; scleroderma; Sjogren's syndrome; cryoglobulinemia; Crohn's disease; ulcerative colitis; autoimmune hepatitis; sclerosing cholangitis; primary biliary cirrhosis; autoimmune pneumonitis; autoimmune cerebritis; thyroiditis; graft versus host disease; Myasthenia gravis; pemphigus vulgaris; temporal arteritis; polymyalgia rheumatica; autoimmune hemolytic anemia; idiopathic thrombocytopenic purpura; thrombotic thrombocytopenic purpura; hemolytic uremic syndrome; Sweet's syndrome; polyarteritis nodosa; microscopic polyarteritis nodosa; amyloidosis; sarcoidosis; familial Mediterranean fever; and the like.

In other embodiments, the therapeutic agent also can be administered to treat congestive heart failure; pain, such as musculoskeletal pain; for weight loss, and the like. The therapeutic agent can be, for example, an anti-inflammatory agent (e.g., aspirin, acetaminophen, ibuprofen, and the like), non-narcotic analgesics (e.g., Tramadol, such as ULTRAM®) and narcotic analgesics (e.g., morphine and morphine derivatives), Sibutramine Hydrochloride Monohydrate (e.g., MERIDIA®), and the like.

In exemplary embodiments, the therapeutic agent can be an immunomodulatory agent, such as, for example, prednisone, methotrexate, soluble TNFα receptor (e.g., ENBREL®), monoclonal antibodies (e.g., REMICADE®), interleukin (cytokine) receptor combinations, neutralizing antibodies, Kineret™ (Anakinra) (an IL-1R antagonist), cyclosporins or ascomycins, or their immunosuppressive analogs (e.g., cyclosporin A, FK-506 (tacrolimus), etc.); rapamycin; corticosteroids; cyclophosphamide; azathioprine; brequinar; leflunamide; mizoribine; deoxyspergualin; analogues thereof, and immunosuppressive monoclonal antibodies, such as, for example, monoclonal antibodies to leukocyte receptors (e.g., MUC, CD2, CD3, CD4, CD7, CD25, CD28, CTLA4, B7, CD45, or CS58) or their ligands; or other immunomodulatory compounds.

The immunomodulatory agent can also be, for example, a biologic agent useful to treating an autoimmune condition. Suitable biologic agents include, for example, soluble TNFα receptor (e.g., ENBREL®), monoclonal antibodies (e.g., REMICADE®), interleukin (cytokine) receptor combinations, neutralizing antibodies, Kineret™ (Anakinra) (an IL-1R antagonist), interferons (e.g., interferon α and λ and analogs thereof). In additional embodiments, the therapeutic agent is not a serotonin agonist or MAO inhibitor.

Dosage Forms

The sleep restorative agent and therapeutic agent can be administered in any unit dosage form, and can be administered in the same dosage form, or in separate dosage forms. Suitable dosage forms include, for example, plain or coated tablets, capsules, lozenges, powders, solutions, suspensions, emulsions, injectable solutions, syrups, suppositories, inhaler, transdermal patches, and the like. The sleep restorative agents can be administered orally, parenterally, sublingually, by inhalation spray, rectally, topically, and the like. The dosage form can contain conventional nontoxic pharmaceutically acceptable carriers, adjuvants, vehicles, and the like, as desired. Topical administration can also involve the use of transdermal administration such as transdermal patches or ionophoresis devices. The term “parenteral” as used herein includes subcutaneous injections, intravenous, intramuscular, intrasternal injection, or infusion techniques. Methods of preparing suitable dosage forms are known, or will be apparent, to those skilled in this art. (See, e.g., Remington's Pharmaceutical Sciences, Mack Publishing Company, Easton, Pa., (1985); which is incorporated by reference herein.)

Injectable preparations, for example, sterile injectable aqueous or oleagenous suspensions can be formulated according to methodologies known in the art using suitable dispersing or wetting agents and suspending agents. The sterile injectable preparation can also be a sterile injectable solution or suspension in a nontoxic parenterally acceptable diluent or solvent, for example, as a solution in ⅓-propanediol. Among the acceptable vehicles and solvents that can be employed are water, Ringer's solution, and isotonic sodium chloride solution. In addition, sterile, fixed oils are conventionally employed as a solvent or suspending medium. For this purpose any bland fixed oil can be employed including synthetic mono- or di-glycerides. In addition, fatty acids such as oleic acid find use in the preparation of injectables.

Because of their ease in administration, tablets and capsules represent an advantageous oral dosage unit form, in which case solid pharmaceutical carriers are employed. Solid dosage forms for oral administration can include capsules, tablets, pills, powders, and granules. In such solid dosage forms, the active compound can be admixed with at least one inert diluent such as sucrose lactose or starch. Such dosage forms can also include additional substances other than inert diluents (e.g., diluents, granulating agents, lubricants, binders, disintegrating agents, and the like). In the case of capsules, tablets, and pills, the dosage forms can also include buffering agents. Tablets and pills can additionally be prepared with sugar or enteric coatings or other pharmaceutically acceptable coatings.

Pramipexole (2-amino-4,5,6,7-tetrahydro-6-(propylamino)benzo-thiazole) is currently available from Pharmacia & Upjohn under the trademark MIRAPEX® in a tablet form for oral administration. The tablets typically contain 0.125 mg, 0.25 mg, 1.0 mg, 1.25 mg or 1.5 mg of (S)-2-amino-4,5,6,7-tetrahydro-6-(propylamino)benzo-thiazole dihydrochloride monohydrate. The tablets typically contain the following inactive ingredients: lactose hydrous, pregelatinized starch, microcrystalline cellulose, sodium starch glycolate, magnesium stearate, purified water, carnauba wax, hydroxypropyl methylcellulose, titanium dioxide, polyethylene glycol, synthetic iron oxide, and polysorbate 80.

Ropinirole (4-[2-(dipropylamino)-ethyl]-1,3-dihydro-2H-indol-2-one mono hydrochloride) is currently available from Smith Kline Beecham under the trademark Requip® in a tablet form for oral administration. The tablets typically contain 0.25 mg, 0.5 mg, 1.0 mg, 2.0 mg or 5.0 mg of 4-[2-(dipropylamino)-ethyl]-1,3-dihydro-2H-indole-2-one monohydrochloride. The tablets typically contain the following inactive ingredients: croscarmellose sodium, hydrous lactose, magnesium stearate microcrystalline cellulose, and one or more of the following: FD&C Blue No. 2 aluminum lake, hydroxypropyl methylcellulose, iron oxides, polyethylene glycol, polysorbate 80, talc, and titanium dioxide.

Zolpidem, as Zolpidem Tartrate, is currently available under the trademark AMBIEN® from G.D. Searle & Co., Chicago, Ill. Zaleplon is currently available under the trademark SONATA®. Both are available in 5 and 10 milligram doses.

Liquid dosage forms for oral administration can include pharmaceutically acceptable emulsions, solutions, suspensions, syrups, and elixirs containing inert diluents commonly used in the art, such as water. Such compositions can also include adjuvants, such as wetting agents, emulsifying and suspending agents, and sweetening, flavoring, and perfuming agents.

Suppositories for rectal administration of the drug can be prepared by mixing the drug with a suitable nonirritating excipient such as, for example, cocoa butter and polyethylene glycols which are solid at ordinary temperatures but liquid at the rectal temperature and will therefore melt in the rectum and release the drug.

The sleep restorative agents can also be administered in the form of liposomes. Liposomes can be derived from phospholipids or other lipid substances. Liposomes can be formed by mono- or multi-lamellar hydrated liquid crystals that are dispersed in an aqueous medium. Any non-toxic, physiologically acceptable and metabolizable lipid capable of forming liposomes can be used. The sleep restorative agents in liposome form can contain, for example, stabilizers, preservatives, excipients, and the like. The typical lipids are phospholipids and phosphatidyl cholines (lecithins), both natural and synthetic. Methods to form liposomes are known in the art. (See, e.g., Prescott (ed.), Methods in Cell Biology, Volume XIV, Academic Press, New York, N.Y. (1976), p. 33 et seq.)

In certain embodiments, the sleep restorative agent and the therapeutic agent can be combined in a solid unitary dosage form, such as a tablet, capsule or pill, thus obviating the need for separate administration of these agent. The combined dosage form can include conventional pharmaceutical carriers or excipients, and, in addition, can include other pharmaceutical agents. Thus, the unit dosage form optionally can be compounded with conventional carriers such as, for example, pharmaceutical grades of mannitol, lactose, starch, magnesium stearate, talcum, cellulose, glucose, sucrose, magnesium carbonate, and the like. Such compositions can contain about 30-90% of active ingredients, typically about 50-90%.

In other embodiments, the sleep restorative agent and therapeutic agent can be administered as separate dosage forms. For example, the sleep restorative agent can be administered as an oral dosage form (e.g., a tablet or pill) while the therapeutic agent can be administered as an injectable solution. The agents can thus be administered on the same schedule or on different schedules, in accordance with clinically effective modes of administration. Each agent can be, for example, plain or coated tablets, capsules, lozenges, powders, solutions, suspensions, emulsions, injectable solutions, syrups, suppositories, inhaler, transdermal patches, and the like, and can be formulated for administration orally, parenterally, sublingually, by inhalation spray, rectally, topically in dosage unit formulations, and the like. Each dosage form can include any suitable carrier, adjuvant, vehicle, excipient, and the like.

Co-Administration of the Therapeutic Agent and Sleep Restorative Agent

In another aspect, co-administration of the therapeutic agent and the sleep restorative agent improves the efficacy of the therapeutic agent. Co-administration refers to the administration to the subject of at least one therapeutic agent and at least one sleep restorative agent. These agents can be administered in the same unit dosage form or in separate dosage forms, and can be administered simultaneously or at different times. The therapeutic agent is typically administered according to any clinically effective mode of administration, as will be appreciated by the skilled artisan.

The sleep restorative agent can be administered according to an effective mode of administration for that agent. Typically an effective amount of the sleep restorative agent is administered to the subject to result in the clinically determinable improvement in the efficacy of the therapeutic agent. As used herein, an “effective amount” refers to an amount of an agent effective to result in clinically determinable improvement in or reduction of one or more symptoms of a disease or condition. For example, effective amounts of the sleep restorative agents of the general formulae (I) and/or (II) can range from about 0.1 mg/day to about 50 mg/day, or from about 0.25 mg/day to about 40 mg/day. The amounts of the sleep restorative agents of general formulae (I) and/or (II) can also range from about 0.5 mg/day to about 20 mg/day.

In one embodiment, the subject is administered an effective amount of 2-amino-4,5,6,7-tetrahydro-6-(propylamino)benzo-thiazole dihydrochloride monohydrate, the (−)-enantiomers thereof, pharmacologically acceptable salts thereof, alone or in association with a pharmaceutically acceptable carrier. The sleep restorative agent can be Pramipexole, such as, for example, (S)-2-amino-4,5,6,7-tetrahydro-6-(propylamino)benzo-thiazole dihydrochloride monohydrate, available from Pharmacia & Upjohn under the trademark MIRAPEX®. MIRAPEX® can be administered, for example, at 0.25 milligram per os (po) qhs for 7 days, then increased by 0.25 mg qweek or as tolerated up to 15 mg qhs, or until the effectiveness of a co-administered therapeutic agent is increased, as desired.

In another embodiment, the subject can receive an effective amount of 4-[2-(dipropylamino)-ethyl]-1,3-dihydro-2H-indol-2-one, or pharmacologically acceptable salts thereof, alone or in association with a pharmaceutically acceptable carrier. The sleep restorative agent can be Ropinirole, 4-[2-(dipropylamino)-ethyl]-1,3-dihyro-2H-indol-2-one, available from Smith Kline Beecham under the trademark REQUIP®. For example, REQUIP® can be administered at 0.25 mg qhs for 7 days, then increased by 0.25 mg qweek or as tolerated to 30 mg qhs, or until the effectiveness of a co-administered therapeutic agent is increased, as desired.

In another embodiment, the sleep restorative agent is Lorazepam, and the pharmacologically acceptable salts thereof, alone or in association with a pharmaceutically acceptable carrier. For example, Lorazepam can be administered at 1-2 mg qhs with Clonazepam at 1-2 mg qhs for four days. Optionally, after four days, Trazodone can be administered at 25 mg q hs and increased as tolerated qhs up to 300 mg q hs. If the subject is groggy, carisoprodol (e.g., about 350-700 mg) or other muscle relaxants or melatonin (e.g., about 3-15 mg qhs) can be substituted for Trazodone.

Lorazepam or Clonazepam optionally can also be co-administered with Pramipexole, Ropinirole, and/or PERMAX®. Trazodone, other muscle relaxants or melatonin can optionally be concurrently administered.

The sleep restorative agent can be Tizanidine (e.g., Zanaflex®), which is administered, for example, at 2-4 mg qhs and increased by 2-4 mg qweek or as tolerated up to 20 mg qhs, or until the effectiveness of a co-administered therapeutic agent is increased, as desired. Tizanidine administration can be supplemented with Pramipexole, Ropinirole, Lorazepam or Clonazepam for racing thoughts, and/or Trazodone, carisoprodol, other muscle relaxants or melatonin to extend sleep to 8 hours.

The sleep restorative agent can also be Gabapentin, which is sold under the trademark NEURONTIN®. For example, Gabapentin can be administered at 300 mg qhs for 3 days, then increased to q3d or as tolerated up to 4800 mg qhs, or until the effectiveness of a co-administered therapeutic agent is increased, as desired. Optionally, Gabapentin can be supplemented with Pramipexole, Ropinirole, Lorazepam or Clonazepam for racing thoughts, and/or Trazodone, carisoprodol, other muscle relaxants or melatonin to extend sleep to 8 hours.

Zolpidem, as Zolpidem Tartrate, is currently available under the trademark AMBIEN® from G.D. Searle & Co., Chicago, Ill. Zaleplon is currently available under the trademark SONATA®. Both are available in 5 and 10 milligram doses, and can be administered, for example, in daily doses of from about 5 to about 30 milligrams per day.

The specific dose level for any particular subject will depend upon a variety of factors, including the activity of the specific sleep restorative agent employed, the age, body weight, general health, sex, diet, time of administration, route of administration, rate of excretion, drug combination(s), the severity of symptoms, and the like.

In general the dosage of a sleep restorative agent can be increased gradually from a starting dose of about 0.1 mg of sleep restorative agent per day and then increased about every 3-7 days to a maximum dose per day as tolerated by the subject and/or as needed to increase the efficacy of the therapeutic agent. Providing subjects do not experience intolerable side effects, the dosage can be titrated to achieve a maximal therapeutic effect. The exact optimal dosage for administration to a subject will vary depending upon which sleep restorative agent is being used. Further, the determination of an optimal dosage requires only routine testing regimes similar to those disclosed herein.

Uses

Typically, an effective amount of the sleep restorative agent is administered to the subject to result in clinically determinable improvement in efficacy of the therapeutic agent. The efficacy of the therapeutic agent can be increased, for example, by decreasing the amount of the therapeutic agent required to be effective to reduce one or more symptoms in the subject receiving the therapeutic agent, to improve the sleep quality of the subject, to reduce sleep disruption, to reduce increased or excessive sympathetic tone, and the like. In certain embodiments, administration of the sleep restorative agent spares (e.g., reduces) the amount of the therapeutic agent that is administered to achieve a comparable reduction of the symptom(s), as compared with a subject receiving the same therapeutic agent, but not the sleep restorative agent.

The sleep restorative agents can also decrease undesirable side effects associated with administration of the therapeutic agent(s). Many therapeutic agents can have toxic side effects when administered at high doses. For example, prednisone administration can be associated with many undesirable side effects, such as, serious infection, osteoporosis, secondary fractures, diabetes, neuropathy, retinopathy, premature death, atherosclerosis, hypertension, bruising, poor wound healing, obesity, fluid retention, edema, hypertension, insomnia, reactivation of TB, and the like. Methotrexate administration can be associated with undesirable side effects, such as, for example, serious infection, bone marrow disease, liver disease, rare death, bone marrow factor insufficiency, poor wound healing, opportunistic and other infections, and the like. Undesired side affects associated with interferon (e.g., IMMUNERON®) and cyclosporine administration include, for example, bone marrow toxicity, liver toxicity, cancer and the like. For leflunomide administration, undesired side effects include, for example, hepatitis, bone marrow toxicity, diarrhea, and the like.

Administration of a sleep restorative agent can allow the amount of the therapeutic agent to be decreased, thereby reducing undesirable (e.g., toxic) side effects. For example, in combination therapy (i.e., when a subject is treated with multiple therapeutic agents) the dose of one therapeutic agent can be reduced or eliminated by administration of a sleep restorative agent. For example, co-administration of a sleep restorative agent with a soluble TNFα receptor can reduce the requirement for secondary immunosuppressants (e.g., prednisone or methotrexate).

The subject is typically monitored (e.g., by a physician) while the therapeutic agent and the sleep restorative agent are administered to the subject. The subject can be a mammal, such as a human or primate. The subject can also be a non-human mammal. As the efficacy of the therapeutic agent increases, the amount of the therapeutic agent required to be administered to the subject is typically decreased (i.e., spared). In some embodiments, the amount of the therapeutic agent administered to the subject can be reduced by 25%, 50%, 75% or more, as compared with a comparable subject receiving the therapeutic agent, but not the sleep restorative agent. In certain embodiments, administration of the therapeutic agent can be discontinued, following a course of administration of the sleep restorative agent. In related embodiments, where more than one therapeutic agent is administered, the administration of at least one of the therapeutic agents can be discontinued following a course of administration of the sleep restorative agent. (See, e.g., infra.)

Co-administration of the therapeutic agent and the sleep restorative agent can reduce or suppression at least one symptom of a disease or condition in the subject. For example, the symptom can be a reduction in pain, such as musculoskeletal pain. An improvement in musculoskeletal pain can be a reduction in intensity and/or frequency of musculoskeletal pain. In some cases, the improvement can be a complete cessation of musculoskeletal pain for a sustained period.

In certain embodiments, the efficacy of the therapeutic agent is increased by improving the sleep quality of the subject. The term “sleep quality” refers to the ability of sleep to refresh a subject. In determining sleep quality, a number of different parameters are considered, including: whether the subject awakes refreshed; the number of sleep interruptions or disruptions; the occurrence of racing thoughts, restlessness, talking in the sleep and/or nightmares; the amount of wake time; the amount of sleep time; the amount of REM sleep; sleep latency; the presence of sleep apnea; teeth grinding; and the like.

The sleep restorative agent can, for example, restore or prolong stage III/Iv sleep in the subject, reduce sleep fragmentation or disruption (i.e., frequent waking during sleep), reduce sleep apnea and/or reduce restless, racing thoughts, talking in one's sleep, nightmares, teeth grinding, restless leg syndrome, the amount of wake time, the amount of sleep time, the amount of REM sleep, sleep latency, and the like. The sleep quality of a subject can be assessed by numerous protocols or procedures, as are known in the art. In one embodiment, sleep quality is assessed through the following questions:
1. Do you wake refreshed?
2. When do you go to bed?
3. When do you wake up?
4. How many times do you wake up and why?
5. Do you have racing thoughts, restlessness, talk in your sleep, or nightmares?
6. Do you snore or pause while breathing while sleeping?
7. Do you grind your teeth?
8. Are you fatigued or sleepy the next day or afternoon?
9. How long have you had these sleep problems?
10. What remedies have you tried?

These questions screen the subject for poor sleep quality, sleep fragmentation, sleep disruption, sleep apnea and restlessness. It is typically noted that a subject that reports refreshing sleep (i.e., awaking feeling refreshed each morning) correlates with the absence of sleep problems or sleep disruption. Because most human subjects over-estimate their sleep quality, administration of a sleep restorative agent to improve sleep quality can be effective even for subjects that report refreshing sleep.

Typically, as the sleep quality of the subject is improved, the efficacy of the therapeutic agent increases. As the efficacy of the therapeutic agent increases, an effective amount decreases (i.e., a smaller amount of the therapeutic agent is required to achieve a similar or comparable reduction of one or more symptoms).

In some embodiments, the subject has a sleep disturbance, which can be overt, characterized by, for example, restless leg syndrome, nightmares, racing thoughts, talking while sleeping, the absence of Stage III/IV sleep, teeth grinding, and the like. The sleep disturbance can also be subtle, characterized by non-restorative sleep upon wakening. Non-restorative sleep is readily diagnosed using, for example, a standardized non-restorative sleep survey, as described above.

The sleep restorative agent also can be administered to a subject whose symptoms are only partially controlled by administration of a therapeutic agent, such as immunomodulatory agent. The sleep restorative agent can be administered to the subject to improve sleep quality, as discussed above. Typically, as the sleep quality of the subject is improved, the efficacy of the immunomodulatory agent increases. In certain embodiments, administration of the sleep restorative agent spares the amount of the immunomodulatory agent required. The sleep restorative agent can be administered to the subject at the initiation of immunomodulatory therapy or later during treatment. In a related embodiment, the sleep restorative agent can be administered to a subject that is, or has become, non-responsive or refractory to the therapeutic agent, to increase the effectiveness of the therapeutic agent.

In a related embodiment, administration of the sleep restorative agent and the therapeutic agent can be synergistic. For example, the combined effect of the sleep restorative agent and the therapeutic agent(s) can be greater than the sum of their individual effects on a subject.

In another aspect, the sleep restorative agent can be administered to a subject exhibiting symptoms of fibromyalgia, as well as another condition for which administration of a therapeutic agent is desired. Symptoms of fibromyalgia can include, for example, musculoskeletal pain symptoms, pain, stiffness, general fatigue, and sleep abnormalities including diminished stage IV sleep. Generalized musculoskeletal pain can be localized at one or more of at least 18 defined characteristic fibromyalgia “tender points” when finger pressure of about 4 kilograms is applied to the area, which test is known as the “tender point index”. As used herein the term “musculoskeletal pain” refers to pain associated with one or more of the 18 defined “tender points” commonly surveyed in the diagnosis of fibromyalgia. The “tender points” survey is well known in the art. (See e.g., Wolfe et al., Arthritis and Rheumatism 33:160-72 (1990).)

The sleep restorative agent also can reduce sympathetic tone in the subject. High sympathetic tone is essentially a stress response, and can be manifested as racing thoughts, restless leg syndrome, nightmares, rapid dreams, busyness at night, and the like. As a basic survival instinct, a “fight or flight response,” it inhibits deep sleep. This primitive fight-or-flight response appears to be controlled by the dopamine-2 receptor family and is aggravated by stimulants, including anxiety, pain, stress, post traumatic stress syndrome, bipolar disorder, caffeine, and the like. Increased sympathetic tone affects sleep, perspiration, gastric acidity, bowel motility, heart rate and temperature control and blood flow through vascular tone. Injured peripheral sympathetic nerves dramatically alter regional blood flow and temperature in Reflex Sympathetic Dystrophy. Overactive central sympathetic tone not only inhibits sleep, but changes peripheral capillary blood flow. Dilated capillaries near the joint may increase tissue porosity or permeability.

Sympathetic tone, and increased or excessive sympathetic tone, in a subject can be determined by a variety of means, including, for example, heart rate variability analysis (see e.g., Mannelli et al., Clin. Exp. Hypertens 19:163-69 (1997); Manuel Martinez-Lavin et al., Arthritis & Rheumatism 41; 1966-71 (1998); Aksoyek et al., J. Auton. Nerv. Syst. 77:190-94 (1999); Penzel et al., Stud. Health Technol. Inform. 77:1256-60 (2000); Orr et al., Am. J. Gastroenterol. 95:2865-71 (2000); U.S. Pat. Nos. 5,921,940 and 5,842,997; the disclosures of which are incorporated by reference herein).

Effective control of sympathetic tone can lead to restoration of a normal sleep pattern, including restorative deep sleep. Restoration of the normal sleep pattern can increase efficacy of therapeutic agents. Similarly, restoration of normal sleep patterns can lead to decreased pain, fatigue, muscular spasm, and other symptoms that can increase sympathetic tone.

For example, soluble TNFα receptor inhibits lymphocyte migration into the target joint, but is not believed to inhibit lymphocyte function. Without intending to be bound by any particular theory, sleep disturbance may interfere with soluble TNFα receptor efficacy by increasing lymphocyte traffic and access to joints and surrounding tissue through dilated capillaries. If lymphocytes can still access the joint despite the presence of soluble TNFα receptor, then lymphocyte function can still injure the joints and adjacent tissues. Thus, lymphocyte function must still be inhibited by prednisone, methotrexate, etc. By administering a sleep restorative agent to decrease sympathetic tone, the porosity of the capillaries is decreased, thereby reducing lymphocyte access to joints and surrounding tissue. The need for agents that inhibit lymphocyte function is similarly reduced.

Similarly, administration of sleep restorative agent(s) can similarly increase the efficacy of other therapeutic agents. For example, therapeutic agents, and therapeutic regimens, that are affected by sleep, perspiration, gastric acidity, bowel motility, heart rate and temperature control and blood flow through vascular tone can have reduced efficacy in a subject with increased or excessive sympathetic tone. Reducing sympathetic tone can decrease the antagonistic effects of these manifestations on therapeutic agents.

The following examples are provided merely as illustrative of various aspects of the invention and shall not be construed to limit the invention in any way.

### EXAMPLES

### Example 1

The effect of sleep disturbance and/or overt fibromyalgia (FM) on Etanercept (ENBREL®, Immunex) efficacy was monitored in an open-label, retrospective chart analysis of rheumatoid arthritis (Ra) and Psoriatic arthritis (PsA) patients starting Etanercept due to inadequate response to prior disease-modifying and steroid treatment. Sixty six (66) consecutive patients (19 males, 47 Females; 39 positive for rheumatoid factor for rheumatoid arthritis, 13 negative for rheumatoid factor for rheumatoid arthritis, 14 having PsA) were treated at a suburban, referral-based rheumatology clinic. The patients were monitored for swollen/tender joints (joint count, JC), fibromyalgia tender point score, restorative sleep quality, and concomitant medication use, before and after adding Etanercept treatment. Concomitant medication use included prednisone, methotrexate, non-steroidal anti-inflammatory drugs (NSAIDS), azothioprine, sulfasalazine, and hydroxychloroquine.

All patients with overt FM and non-restorative sleep were treated with options for fibromyalgia (FM), including Trazodone, Lorazepam, Clonazepam, Carisoprodol, and/or Pramipexole as needed to decrease FM pain and sleep disturbance as previously reported. (Arthritis & Rheumatology 43:9 A1599 (2000); Arthritis & Rheumatology 42:9 A385 (1999); Arthritis & Rheumatology 41:9 A1359 (1998).)

The patient criteria were as follows: The mean arthritis duration was 9.2±1.3 years. Etanercept use was 14±1.5 months. The average age of the patients was 50.5±4.3 years. Their average initial erythrocyte sedimentation rate (ESR) was 22.0±8.0. Previous DMARDs 2.4±0.3. Their initial Joint Count was as follows: PsA 8.3, Ra-8.1, and Ra+13.2 The disease groups were combined: 20 patients had active FM (aFM). 29 never had FM (no FR). 17 had inactive FM (iFM). The patient characteristics are summarized in the following tables.

Patient Characteristics

| | RA+(39) | Ra−(13) | PsA(14) |
|---|---|---|---|
| AGE | 50.9 | 51.0 | 50.5 |
| Yrs of arthr. | 10.6 | 8.8 | 8.0 |
| DMARDs | 2.6 | 1.6 | 2.1 |
| ESR | 32.1 | 13.9 | 22.5 |
| Swollen Joints | 13.2 | 8.1 | 8.3 |
| MTX Dose(mg/wk) | 16.5 | 17.9 | 23.8 |
| Pred. Dose (mg/d) | 9.0 | 5.9 | 10.7 |
| Etanercept (mo.) | 12.8 | 11.9 | 14.7 |

Patient Characteristics

| | No FM (29) | iFM (17) | aFM (20) |
|---|---|---|---|
| Age | 51.4 | 52.6 | 48.5 |
| Yrs of Arthr. | 10.3 | 9.9 | 8.6 |
| DMARDs | 2.0 | 2.4 | 2.6 |
| ESR | 29.0 | 16.6 | 30.8 |
| Swollen Joints | 11.6 | 10.1 | 11.5 |
| MTX Dose (mg/wk) | 19.1 | 19.7 | 15.0 |
| Pred Dose (mg/d) | 8.0 | 7.3 | 10.3 |
| Etanercept (mo.) | 11.9 | 15.4 | 12.6 |

The following table summarizes the results of the study

| Variable | No FM | iFM | aFM |
|---|---|---|---|
| Δ Joint Count | 11.6 → 0.7 | 10.1 → 0.5 | 11.5 → 5.9 * |
| Δ Pred. (mg/d) | 8.0 → 0.3 | 7.3 → 0.4 | 10.3 → 5.9 # |
| Δ MTX (mg/wk) | 19.1 → 2.0 | 19.7 → 0.0 | 15.0 → 13.3 ** |

M-W P value:
* = 0.066,
# = 0.1,
** = 0.002

As can be seen from these results, administering a sleep restorative agent (e.g., Pramipexole, Gabapentin, Clonazepam, Lorazepam, Trazodone, or other night time anti-depressant, muscle relaxant or melatonin) dramatically decreased the effective dosages for prednisone and methotrexate. This decrease was most dramatic for patients without fibromyalgia, or with inactive fibromyalgia. For patients with the active fibromyalgia, the decrease in effective dosages was also significant. The following table presents the data as a percentage decrease in joint count, or as a percentage decrease in medication (i.e., therapeutic agent).

| % Change | No FM | iFM | aFM |
|---|---|---|---|
| Joint Count | −93.9 ± 1.9% | −95.0 ± 1.9% | −47.8 ± 14.% * |
| Prednisone Dose | −96.3 ± 4.0% | −94.5 ± 3.6% | −48.5 ± 15.% * |
| Methotrexate Dose | −89.5 ± 8.4% | −100. ± 0.0% | −11.3 ± 4.5% # |

M-W P value:
* = 0.001,
# < 0.001

Administration of a sleep restorative agent also allowed a decrease in the amounts of other therapeutic medications administered to the patients, as summarized in the following table:

| Medication Discontinued | No FM | iFM | aFM | p value |
|---|---|---|---|---|
| Azathioprine/ | 10/11 | 9/9 | 4/8 | 0.019 |
| Sulfasalazine/ | | | | |
| Hydroxychloroquine | | | | |
| NSAIDs | 6/7 | 4/7 | 1/13 | 0.045 |

An analysis of the data shows that a variety of sleep restorative agents were effective in treating symptoms of Ra or PsA in patients having inactive fibromyalgia and in those with active fibromyalgia, as summarized in the following table.

| Restlessness | iFM (17) | aFM (20) |
|---|---|---|
| Carbidopa-Levodopa 25/100 | 0 | 1 |
| Lorazepam 1-2 mg | 4 | 7 |
| Clonazepam 1-2 mg | 3 | 4 |
| Gabapentin 300-1200 mg | 1 | 2 |
| Pramipexole 0.25-2 mg | 3 | 4 |

In contrast, conventional sleep inducing agents, when used alone, were less effective in treating symptoms of Ra or PsA in patients having inactive fibromyalgia and in those with active fibromyalgia, as summarized in the following table.

| | |
|---|---|
| iFM (17) | aFM (20) |

Deep Sleep Inducing Agents

| | | |
|---|---|---|
| Amitriptyline | 0 | 2 |
| Nortriptyline | 1 | 0 |
| Doxepin | 0 | 1 |
| Cyclobenzaprine | 1 | 0 |
| Melatonin | 1 | 1 |

Other

| | | |
|---|---|---|
| Sertraline | 1 | 3 |
| Resperdal | 0 | 1 |
| Refused Treatment | 0 | 3 |

Thus, for all patients in the study, administering a sleep restorative agent improved Etanercept efficacy for treatment of rheumatoid arthritis and Psoriatic arthritis, and patients also decreased other medications including NSAID (p=0.045) and other DMARDs (p=0.019).

### Example 2

The study of Example 1 was continued to 18 months. For these subjects, at 12 months, 20 patients with autoimmune disease had active fibromyalgia (FM). Fibromyalgia was a surrogate for a lack of deep (e.g., stage IV) restorative sleep. After active management of these cases to address FM/sleep concerns, at 18 months there were only 10 patients with active FM. Most of these patients had just non-restorative sleep, rather than FM tender points. One patient without FM, developed FM, and now needs prednisone 10 mg qd. this patient will be treated with pramipexole to convert her back to inactive FM, which is expected to allow her to discontinue prednisone.

Data at 18 Months

| | (N) No FM | (N) Inactive FM | (N) Active FM | p value |
|---|---|---|---|---|
| Joint | (28) −11.54 | (23) −8.98 | (10) −10.95 | 0.5685 |
| count | | | | |
| abs | | | | |
| MTX | (16) −17.5 mg | (9) −20.1 mg | (6) +0.8 mg | 0.0002 |
| abs | | | | |
| Pred | (19) −7.32 mg | (13) −7.92 mg | (9) −7.15 mg | 0.8649 |
| abs | | | | |

The p values and absolute decreases (abs) in steroid and methotrexate use demonstrate the sparing effect of sleep restorative agents. Generally, patients who slept normally or received an agent according to the present invention (as opposed to sedative hypnotics or antidepressants) no longer have swollen, tenderjoints or use prednisone or steroids after Etanercept was added to their treatment. Generally, the remaining symptom in some patients was non-restorative sleep, was correlated with a continued requirement of Methotrexate.

The data for attempted conversion of active-FM patients (at 12 months) to inactive-FM patients at 18 months is shown below. #1 refers to patient status at study entry. #2 refers to patient status at 12 months. #3 refers to patient status at 18 months. JC refers to the number of swollen tender joints. MTX stands for methotrexate, and Pred stands for prednisolone; this data is reported as mg of drug used at each time point.

Patients Converted From Active FM to Inactive FM

| | (# tender joints) | | | (mg dose) | | | (mg dose) | | |
|---|---|---|---|---|---|---|---|---|---|
| Patient # | JC1 | JC2 | JC3 | MTX1 | MTX2 | MTX3 | Pred1 | Pred2 | Pred3 |
| 1 | 3 | 1 | 0 | | | | 15 | 0 | 0 |
| 2 | 12 | 3 | 1 | | | | | | |
| 3 | 10 | 18 | 0 | | | | 20 | 10 | 0 |
| 4 | 8 | 0 | 0 | | | | 5 | 10 | 0 |
| 5 | 16 | 0 | 0 | | | | 10 | 5 | 8 |
| 6 | 15 | 10 | 0 | 20 | 0 | 0 | 10 | 5 | 0 |
| 7 | 0 | 0 | 0 | 15 | 0 | 0 | 3 | 3 | 3 |
| Mean | 9.1 | 4.6 | 0 | 17.5 | 0 | 0 | 10.5 | 5.5 | 1.8 |

Dates for Patients Who Converted From No FM to Active FM

| Patient # | JC1 | JC2 | JC3 | MTX1 | MTX2 | MTX3 | Pred1 | Pred2 | Pred3 |
|---|---|---|---|---|---|---|---|---|---|
| 1 | 22 | 0 | 10 | 12 | 0 | 0 | 0 | 0 | 10 |

Generally, the improved patients adding pramipexole, lorazepam and/or clonazepam to their treatment did much better from the 12 month time point to the 18 month time point. They still improved initially, but their response was much greater later.

### Example 3

Three patients with dry eyes and mouth from Sjogren's Syndrome, were treated with lorazepam or pramipexole. The dryness had previously been partially controlled with Hydroxychloroquine 200 mg po twice a day (bid) for many years. Lorazepam was administered at 1-2 mg po qhs. Pramipexole was later administered at 0.5-4.0 mg qhs. Complete resolution of the dryness in 2 of 3 patients was observed, and nearly complete resolution of dryness in the third patient. One daily dose of Hydroxychloroquine was discontinued and benefit maintained.

### Example 4

Three patients with Behcet's Syndrome were treated according to the present invention. For one patient, prednisone administration was supplemented with lorazepam at 2 mg qhs. The patient was able to d/c prednisone in two months after controlling skin manifestations of Behcet's Syndrome. For a second patient, lorazepam at 2 mg qhs was co-admnistered with Dapsone and prednisone. Following such treatment, Dapsone could be discontinued without further seizures. When pramipexole was added, prednisone 5 mg qd could be discontinued. No fatigue, mouth ulcers, skin vasculitis, hoarse voice or seizures were observed in the patient. For the third patient, clonazepam was added at 2 mg qhs, and less fatigue and no further mouth ulcers were observed in the patient. This patient was able to discontinue prednisone 10 mg qd.

### Example 5

Six patients with Ankylosing Spondylitis were treated according to the present invention. All six patients had been treated with Methotrexate, steroids 2-10 mg qd and Etanercept for many years. The patients were administered lorazepam 1-2 mg qhs or pramipexole 0.5-3.0 mg qhs. All six patients were able to discontinue Methotrexate and steroids. Etanercept administration was continued, but without evidence of active disease.

### Example 6

Patients with Systemic Lupus Erythematosis were treated according to the present invention. A woman hospitalized for life threatening leukopenia was forced to take steroids 60 mg qd and hydroxychloroquine 200 mg bid for three months, without improvement in symptoms. Heart Variability Analysis (HVA) demonstrated this patient had increased sympathetic activity consistent with her poor sleep, racing thoughts, disruptive dreams and stress. Pramipexole was added and increased to 3.5 mg q hs over 3 months, During this treatment, this patient was able to decrease prednisone to 5 mg qd; and her HVA showed normal values. She is currently maintaining normal complete blood count.

Another 8 patients with Systemic Lupus Erythematosis were able to decrease or d/c prednisone by 80% by adding pramipexole for to improve sleep and/or decrease sympathetic activity. The dose range of pramipexole ranged from 2-6 mg qhs.

A male patient failed methotrexate and almost died on azathioprine due to bone marrow toxicity. He was steroid-dependent at 20 mg qd on leflunamide. Adding lorazepam 2 mg qhs, this patient could decrease prednisone to 15 mg qhs. Adding pramipexole 3-4.5 mg qhs allowed him to decrease prednisone to 5 mg qd for the first time in 7 years. He noted much less fatigue, anemia, joint pain, and had stable kidney function.

### Example 7

A patient with ocular and articular Sarcoidosis was steroid-dependent for two years. Pramipexole was administered to 3 mg qhs, and the patient was able to decrease steroids 90% and still control the disease.

### Example 8

Five patient with Palindromic Rheumatism were treated according to the present invention. After adding clonazepam 2 mg, pramipexole 1-6 mg and/or lorazepam 2 mg qhs to their treatment, the patients reported that episodic severe pain decreased dramatically, by 90%. The patients subsequently were off narcotics, steroids, Etanercept, Infliximab, leflunamide, Azothioprine, sulfasalazine, hydroxychloroquine and colchicine.

### Example 9

A patient with Reiter's Syndrome, treated with Etanercept, was able to discontinue methotrexate, steroids 5-10 mg qd, and narcotics after dactylitis and synovitis responded better to Etanercept after adding pramipexole up to 4 mg qhs.

### Example 10

A patient with chronic gout, exhibiting severe synovitis, which was uncontrolled by prednisone 10-40 mg qd for two months. Adding colchicine, hydroxychloroquine and Etanercept only allowed him to decrease prednisone to 15 mg qd. Adding pramipexole and lorazepam allowed the patient to discontinue steroids, colchicine, hydroxychoroquine, and narcotics.

### Example 11

A patient with pseudogout, reported no flares for three years on colchicine after adding lorazepam 2 mg qhs for sleep. For this patient, the flare ups were previously uncontrolled on colchicine.

### Example 12

Eight patients with Multiple Sclerosis (MS) were treated according to the present invention. In MS patients, both untreated and on Interferon-Beta-1b (BETASERON®), MS is much more active when fibromyalgia is active. For three patients, when pramipexole and/or lorazepam was added, their MS was quiet for over three years. One patient was flaring uncontrollably. Lorazepam was added at 2 mg qhs, but the patient still exhibited poor sleep and fibromyalgia. This patient was pramipexole and Ropinirole intolerant.

### Example 13

Patients having rheumatoid arthritis and receiving prednisone were able to lower their prednisone doses following administration of Ropinirole 2-20 mg qhs.

### Example 14

Patients having rheumatoid arthritis and receiving Leflunamide were found to exhibit increased Leflunamide efficacy upon administration of lorazepam, clonazepam and/or pramipexole.

### Example 15

Patients having rheumatoid arthritis and receiving Inflixamab exhibited increased efficacy of Infliximab with lorazepam 1-2 mg qhs or with pramipexole.

### Example 16

Heart variability date was determined for patients have rheumatoid arthritis and receiving treatment with a soluble TNFα receptor (i.e., ENBREL®).

| | | | | | | Std. |
|---|---|---|---|---|---|---|
| ENBREL ® | | N | Minimum | Maximum | Mean | Deviation |
| Success | VAGUS | 20 | .07 | .29 | .1765 | .05923 |
| | SYMPATH | 20 | 23 | 72 | 50.60 | 14.103 |
| | TENSION | 20 | 36 | 490 | 202.60 | 141.765 |
| | TPOWER | 20 | 140 | 2340 | 631.90 | 603.119 |
| | Valid N (listwise) | 20 | | | | |
| failure | VAGUS | 21 | .05 | .28 | .1005 | .06160 |
| | SYMPATH | 21 | 41 | 91 | 70.19 | 14.885 |
| | TENSION | 21 | 73 | 1099 | 598.05 | 311.658 |
| | TPOWER | 21 | 34 | 1458 | 258.71 | 337.934 |
| | Valid N (listwise) | 21 | | | | |

ENBREL® was administered in addition to other medication, as necessary, for rheumatoid arthritis. Treatment success was defined by the patients being able to discontinue other medications administered for rheumatoid arthritis, including DMARDs, NSAIDS and steroids. Treatment failure was defined as the inability of the patients to discontinue other medications, due to the persistence of symptoms requiring administration of those medications. Both group exhibited few symptoms of joint pain or evidence of rheumatoid arthritis.

For these two groups of patients, their heart variability data was determined. Four heart variability measurements were found to be predictive, at P<0.001, of success or failure of the treatment protocol. These measurements included vagus tone, total power (which is inversely proportional to sympathetic activity) and Sympathetic and tension index.

Paired Samples Statistics

| | | | | | Std. | Std. Error |
|---|---|---|---|---|---|---|
| ENBREL | | | Mean | N | Deviation | Mean |
| Success | Pair | SYMPATH | 50.60 | 20 | 14.103 | 3.153 |
| | 1 | ENBREL | 1.0000 | 20 | .00000 | .00000 |
| failure | Pair | SYMPATH | 70.19 | 21 | 14.885 | 3.248 |
| | 1 | ENBREL | 2.0000 | 21 | .00000 | .00000 |

Paired Samples Test

| | Paired Differences | | | | |
|---|---|---|---|---|---|
| | | | | 95% Confidence | |
| | | | Std. | Interval of the | |
| | | Std. | Error | Difference | |
| ENBREL | Mean | Deviation | Mean | Lower | Upper |
| Success Pair 1 | 49.600 | 14.10263 | 3.15344 | 42.9998 | 56.2002 |
| SYMPATH-ENBREL | | | | | |
| failure Pair 1 | 68.190 | 14.88496 | 3.24816 | 61.4149 | 74.9660 |
| SYMPATH-ENBREL | | | | | |

The results show that a validated measure of sympathetic tone predicts that those patients with higher sympathetic tone and lower parasympathetic tone require more medication when using ENREL® soluble TNFα receptor for rheumatoid arthritis. Thus, sympathetic tone can be monitored during treatment with sleep restorative agents to effect a decrease in excessive sympathetic tone as an inhibitor of deep sleep.

The previous examples are provided to illustrate but not to limit the scope of the claimed inventions. Other variants of the inventions will be readily apparent to those of ordinary skill in the art and encompassed by the appended claims. All publications, patents, patent applications and other references cited herein are hereby incorporated by reference.

What is claimed is:

1. A method for increasing the efficacy of a therapeutic agent administered to a subject having an autoimmune condition, comprising co-administering to the subject an effective amount of a sleep restorative agent or a pharmacologically acceptable addition salt thereof, and a therapeutic agent;
whereby the efficacy of the therapeutic agent is increased.

2. The method of claim 1, wherein an undesired side effect associated with administration of the therapeutic agent is reduced.

3. The method of claim 1, wherein a symptom of the subject is reduced.

4. The method of claim 1, wherein administration of the sleep restorative agent spares the effective amount of the therapeutic agent.

5. The method of claim 1, wherein sleep quality of the subject is increased.

6. The method of claim 5, wherein increased sleep quality is manifested by restoration or prolongation of stage III/IV sleep, decreased sleep fragmentation or disruption, reduced sleep apnea, reduced restless legs syndrome, decreased restlessness, decreased racing thoughts, decreased talking in one's sleep or decreased nightmares.

7. The method of claim 1, wherein excessive sympathetic tone in the subject is reduced.

8. The method of claim 1, wherein the sleep restorative agent is a compound of the following formula: 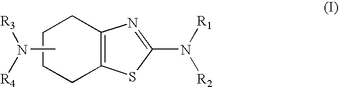
wherein
R₁ represents a hydrogen atom, a C₁₋₆ alkyl group, a C₃₋₆ alkenyl, a C₃₋₆ alkynyl, a C₁₋₆ alkanoyl group, a phenyl C₁₋₃ alkyl group, or a phenyl C₁₋₃ alkanoyl group, wherein the phenyl nuclei may be substituted by 1 or 2 halogen atoms;
R₂ represents a hydrogen atom or a C₁₋₄ alkyl group;
R₃ represents a hydrogen atom, a C₁₋₇ alkyl group, a C₃₋₇ cycloalkyl group, a C₃₋₆ alkenyl group, a C₃₋₆ alkynyl group, a C₁₋₇ alkanoyl group, a phenyl C₁₋₃ alkyl, or a phenyl C₁₋₃ alkanoyl group, wherein the phenyl nucleus may be substituted by fluorine, chlorine or bromine atoms;
R₄ represents a hydrogen atom, a C₁₋₄ alkyl group, a C₃₋₆ alkenyl group, or a C₃₋₆ alkynyl group; or
R₃ and R₄ together with the nitrogen atom between them represent a pyrrolidino, piperidino, hexamethyleneimino or morpholino group.

9. The method of claim 8, wherein, wherein the sleep restorative agent is 2-amino-4,5,6,7-tetrahydro-6-(propylamino)benzo-thiazole or the (−)-enantiomer thereof.

10. The method of claim 1, wherein the sleep restorative agent is a compound of the following formula: 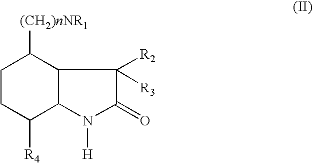
wherein
R₁ is hydrogen or a C₁₋₄ alkyl group;
R₂ and R₃ are each hydrogen or a C₁₋₄ alkyl group;
R₄ is hydrogen or hydroxy; and
n is 1 to 3.

11. The method of claim 10, wherein the sleep restorative agent is 4-[2-(dipropylamino)-ethyl]-1,3-dihydro-2H-indol-2-one.

12. The method of claim 1, wherein the sleep restorative agent is Lorazepam, Clonazepam, Tizanidine, Gabapentin, Zaleplon, Zolpidem, pregabalin, or pharmaceutically acceptable salts thereof.

13. The method of claim 1, wherein the therapeutic agent is soluble TNFα receptor, methotrexate, prednisone, an interferon, a cyclosporin, an ascomycin, a rapamycin, a corticosteroid, a cyclophosphamide, azathioprine, brequinar, leflunomide, mizoribine, deoxyspergualin, or immunosuppressive monoclonal antibodies to a leukocyte receptor.

14. The method of claim 13, wherein the soluble TNFα receptor is Etanercept or Lenercept.

15. The method of claim 1, wherein the sleep restorative agent and the therapeutic agent are administered in a unitary dosage form.

16. The method of claim 1, wherein the sleep restorative agent and the therapeutic agent are administered separately.

17. The method of claim 1, wherein the sleep restorative agent is administered as a dosage form of a tablet, capsule, lozenge, powder, solution, suspension, emulsion, injectable solution, syrup, suppository, or transdermal patch.

18. The method of claim 17, wherein the dosage form further comprises a pharmaceutically acceptable carrier.

19. The method of claim 1, wherein the therapeutic agent is an immunomodulatory agent.

20. A method for sparing an effective amount of a therapeutic agent administered to a subject having an autoimmune condition, comprising:
co-administering to the subject the therapeutic agent and an effective amount of a sleep restorative agent, the sleep restorative agent improving sleep quality of the subject;
whereby the sleep restorative agent spares the effective amount of the therapeutic agent.

21. The method of claim 20, wherein an undesired side effect associated with administration of the therapeutic agent is reduced.

22. The method of claim 20, wherein the autoimmune condition is rheumatoid arthritis; psoriatic arthritis; a spondyloarthropathy; palindromic rheumatism; systemic lupus erythematosus; vasculitis with systemic lupus erythematosus; multiple sclerosis; Hashimoto's thyroiditis; chronic pseudogout; hepatitis C arthritis, mixed connective tissue disease; dermatomyositis, polymyositis; scleroderma; Sjogren's syndrome; cryoglobulinemia; Crohn's disease; ulcerative colitis; autoimmune hepatitis; sclerosing cholangitis; primary biliary cirrhosis; autoimmune pneumonitis; auto immune cerebritis; thyroiditis; graft versus host disease; Myasthenia gravis; pemphigus vulgaris; temporal arteritis; polymyalgia rheumatica; autoimmune hemolytic anemia; idiopathic thrombocytopenic purpura; thrombotic thrombocytopenic purpura; hemolytic uremic syndrome; Sweet's syndrome; polyarteritis nodosa; microscopic polyarteritis nodosa; amyloidosis; sarcoidosis; or familial Mediterranean fever.

23. The method of claim 22, wherein the spondyloarthropathy is Behcet's disease, Whipple's Disease, sarcoidosis, ankylosing spondylitis or Reiter's Syndrome.

24. A method for sparing an effective amount of a therapeutic agent administered to a subject having an autoimmune condition, comprising:
co-administering to the subject the therapeutic agent and an effective amount of a sleep restorative agent, the sleep restorative agent reducing excessive sympathetic tone of the subject;
whereby the sleep restorative agent spares the effective amount of the therapeutic agent.

25. A method for reducing a symptom in a subject in need of immunomodulatory therapy, comprising co-administering an effective amount of an immunomodulatory agent and an effective amount of a sleep restorative agent, the sleep restorative agent improving sleep quality of the subject;
whereby the sleep restorative agent spares the effective amount of the immunomodulatory agent needed to reduce the symptom.

26. The method of claim 25, wherein the immunomodulatory agent is soluble TNFα receptor, prednisone, methotrexate, an interferon, a cyclosporin, an ascomycin, a rapamycin, a corticosteroid, a cyclophosphamide, azathioprine, brequinar, leflunomide, mizoribine, deoxyspergualin, or immunosuppressive monoclonal antibodies to a leukocyte receptor.

27. The method of claim 26, wherein the immunomodulatory agent is soluble TNFα receptor.

28. The method of claim 25, wherein the subject has a sleep disorder.

29. The method of claim 25, wherein a side effect associated with administration of the therapeutic agent is reduced.

30. A composition for administration to a subject having an autoimmune disease, comprising:
an effective amount of a sleep restorative agent; and
and an effective amount of a therapeutic agent;
the effective amount of the therapeutic agent spared by the sleep restorative agent.

31. The composition of claim 30, wherein the composition is a unitary dose.

32. The composition of claim 30, wherein the composition is administered as a tablet, capsule, lozenge, powder, solution, suspension, emulsion, injectable solution, syrup, suppository, or transdermal patch.

33. The composition of claim 30, wherein the composition further comprises a pharmaceutically acceptable carrier, an excipient or an adjuvant.

34. The composition of claim 30, wherein the sleep restorative agent is a compound of the following formula: 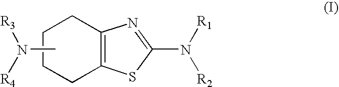
wherein
R₁ represents a hydrogen atom, a C₁₋₆ alkyl group, a C₃₋₆ alkenyl, a C₃₋₆ alkynyl, a C₁₋₆ alkanoyl group, a phenyl C₁₋₃ alkyl group, or a phenyl C₁₋₃ alkanoyl group, wherein the phenyl nuclei may be substituted by 1 or 2 halogen atoms;
R₂ represents a hydrogen atom or a C₁₋₄ alkyl group;
R₃ represents a hydrogen atom, a C₁₋₇ alkyl group, a C₃₋₇ cycloalkyl group, a C₃₋₆ alkenyl group, a C₃₋₆ alkynyl group, a C₁₋₇ alkanoyl group, a phenyl C₁₋₃ alkyl, or a phenyl C₁₋₃ alkanoyl group, wherein the phenyl nucleus may be substituted by fluorine, chlorine or bromine atoms;
R₄ represents a hydrogen atom, a C₁₋₄ alkyl group, a C₃₋₆ alkenyl group, or a C₃₋₆ alkynyl group; or
R₃ and R₄ together with the nitrogen atom between them represent a pyrrolidino, piperidino, hexamethyleneimino or morpholino group.

35. The method of claim 34, wherein, wherein the sleep restorative agent is 2-amino-4,5,6,7-tetrahydro-6-(propylamino)benzo-thiazole or the (−)-enantiomer thereof.

36. The method of claim 30, wherein the sleep restorative agent is a compound of the following formula: 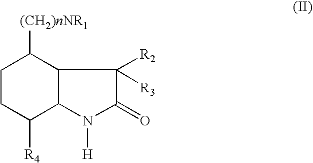
wherein
R₁ is hydrogen or a C₁₋₄ alkyl group;
R₂ and R₃ are each hydrogen or a C₁₋₄ alkyl group;
R₄ is hydrogen or hydroxy; and
n is 1 to 3.

37. The method of claim 36, wherein the sleep restorative agent is 4-[2-(dipropylamino)-ethyl]-1,3-dihydro-2H-indol-2-one.

38. The method of claim 30, wherein the sleep restorative agent is Lorazepam, Clonazepam, Tizanidine, Gabapentin, Zaleplon, Zolpidem, or pharmaceutically acceptable salts thereof.

39. The method of claim 30, wherein the therapeutic agent is soluble TNFα receptor, methotrexate, prednisone, an interferon, a cyclosporin, an ascomycin, a rapamycin, a corticosteroid, a cyclophosphamide, azathioprine, brequinar, leflunomide, mizoribine, deoxyspergualin, or immunosuppressive monoclonal antibodies to a leukocyte receptor.
